(19) United States
(12) Patent Application Publication
(10) Pub. No.: US 2016/0310403 A1
(43) Pub. Date: Oct. 27, 2016

(54) SKIN COSMETIC PRODUCT

(71) Applicant: SHISEIDO COMPANY, LTD., TOKYO (JP)

(72) Inventor: KAZUTAKA SASAKI, YOKOHAMA-SHI (JP)

(73) Assignee: SHISEIDO COMPANY, LTD., TOKYO (JP)

(21) Appl. No.: 15/099,737

(22) Filed: Apr. 15, 2016

Related U.S. Application Data
(60) Provisional application No. 62/153,016, filed on Apr. 27, 2015.

(51) Int. Cl. A61K 8/87; A61K 8/02; A61Q 19/00; A61K 8/85

(52) U.S. Cl. CPC A61K 8/87; A61K 8/85; A61K 8/0216; A61Q 19/00; A61K 2800/26

(57) ABSTRACT

An aqueous skin cosmetic product exerting a high irregularity correction effect, providing a non-greasy and light feeling when applied and having an excellent sebum suppressing effect is provided. The skin cosmetic product of the present invention is characterized by comprising (A) 15 to 50% by mass of a powder formed of an (adipic acid/neopentyl glycol) crosspolymer and (B) 0.5 to 3% by mass of a hydrophobically modified polyurethane. (PEG-240/Decyltetradeceth-20/HDI) copolymer is preferable as the hydrophobically modified polyurethane, It is preferable that the skin cosmetic product is a gel having a viscosity of 15,000 mPa·s or more and 200,000 mPa·s or less.

### CROSS REFERENCE TO RELATED APPLICATIONS

This application relates to and claims priority from U.S. Prov. 62/153,016 filed Apr. 27, 2015, the entire contents of which are fully enclosed by reference

### DRAWING SELECTED FOR PUBLICATION

None

### BACKGROUND OF THE INVENTION

1. Field of the Invention

The present invention relates to a skin cosmetic product which is excellent in irregularity correction effect for the skin such as pores and fine lines and has refreshing feeling when used as well as being excellent in sebum suppressing effect.

2. Description of Related Art

It has been desired to develop cosmetic products making irregularities of the skin (such as pores, fine lines or rough-texture) less noticeable, and various cosmetic products having an irregularity correction effect on the basis of optical properties of a powder have been proposed. These cosmetic products contain a powder component having a light diffusion effect. It is considered that light transmitted through a cosmetic film is diffused by the powder component, which has a light diffusion effect, and a border between light and dark portion of the skin is blurred, and thus irregularities are made less noticeable.

As the powder component to be blended in the cosmetic products having an irregularity correcting effect, for example, titanium dioxide coated mica having coherency of light has been frequently used; however, titanium dioxide coated mica has a problem in that irregularities of the skin are rather made noticeable since light is reflected by the surface of the powder. Then, in an attempt to obtain natural finish, ideas of suppressing surface reflection by coating the surface of a flaky powder base such as titanium dioxide coated mica with various materials or employing silicone-based solid particles have been tested.

JP-A 2013-216602 discloses a cosmetic product containing a coated particle, which is produced by coating the surface of a flaky powder base-material with silicone elastomer particles or silicone elastomer/silicone resin composite particles, and an oily component containing a volatile silicone oil in combination. It is reported that this cosmetic product is designed by approximating the refraction index of the coating layer of the coated particle to that of the oily component, with the result that the skin looks glossy immediately after application, and shiny skin developed with time due to sebum is suppressed and pores are made less noticeable.

Other than JP-A 2013-216602, powders modified in various ways have been proposed; however, powders used in conventional irregularity correction cosmetic products were principally blended in oil. Accordingly, cosmetic products containing a large amount of powder in order to obtain sufficient irregularity correction effect inevitably contain an oil to some extent, with the result that the products often provide heavy feeling to the users when applied.

In the meantime, a powder formed of an (adipic acid/neopentyl glycol) crosspolymer can be used as a wrinkle-concealing component based on its characteristic light scattering property. In addition, it is reported that this powder has a sebum absorbing ability and can be blended both in an oil phase and a water phase (Catalog of “AuraSphere”® by Centerchem Inc.). However, the powder has a problem in that if a large amount of powder is blended particularly in a water phase, it tends to be precipitated. Because of this, the manufacturer recommended that the content is 3% or less (technical material of “Penstia”™ Powder by Centerchem Inc.). For this reason, the irregularity correction effect and sebum suppressing effect due to the powder have not yet been sufficiently obtained.

### SUMMARY OF THE INVENTION

The present invention was made under the aforementioned circumstances. An object of the invention is to provide an aqueous skin cosmetic product exerting a high irregularity correction effect, providing a non-greasy and light feeling when applied and having an excellent sebum suppression effect.

As a result of intensive studies, the present inventors have found that a powder formed of an (adipic acid/neopentyl glycol) crosspolymer can be blended in a large amount in a water phase by adding the powder in combination with a specific aqueous thickening agent, with the result that a high irregularity correction effect and sebum suppression effect can be obtained. Based on the finding, the present invention was accomplished.

More specifically, the present invention provides a skin cosmetic product comprising:

(A) 15 to 50% by mass of a powder formed of an (adipic acid/neopentyl glycol) crosspolymer, and

(B) 0.5 to 3% by mass of a hydrophobically modified polyurethane.

As the hydrophobically modified polyurethane, a (PEG-240/Decyltetradeceth-20/HDI) copolymer is preferable.

The skin cosmetic product according to the present invention can stably comprise a large amount of (adipic acid/neopentyl glycol) crosspolymer powder since a hydrophobically modified polyurethane is blended. As a result, an irregularity correction (pores concealing) effect and a sebum suppressing effect due to the (adipic acid/neopentyl glycol) crosspolymer powder are sufficiently exerted. In addition, since the (adipic acid/neopentyl glycol) crosspolymer powder can be stably blended in a water phase in a large amount, it is not necessary to add a large amount of oil, with the result that non-greasy and watery sense of use intrinsic to an aqueous cosmetic product can be obtained.

The “sebum suppression effect” in the present invention does not mean an effect for suppressing secretion of sebum, but means an effect for suppressing shiny skin due to sebum by absorbing the sebum with the powder.

### BRIEF DESCRIPTION OF THE PREFERRED EMBODIMENTS

### (A) (Adipic Acid/Neopentyl Glycol) Crosspolymer Powder

The (adipic acid/neopentyl glycol) crosspolymer powder to be used in the skin cosmetic product of the present invention is a powder produced by crosslinking a copolymer of adipic acid and neopentyl glycol with isopropyl triethoxysilane.

The shape and particle diameter of the (adipic acid/neopentyl glycol) crosspolymer powder are not particularly limited; however, a spherical powder having an average diameter of usually 1 to 30 μm and preferably about 3 to 20 μm is used.

As the (adipic acid/neopentyl glycol) crosspolymer powder, a commercially available product may be used; for example, “Penstia Powder” (trade name) manufactured by CENTERCHEM Inc. is preferable.

In the skin cosmetic product of the present invention, the content of the (adipic acid/neopentyl glycol) crosspolymer powder is 15% by mass or more, 20% by mass or more, or 25% by mass or more. If the content is less than 15% by mass, for example, about 3% by mass conventionally recommended, a sufficient irregularity correction effect and sebum suppressing effect cannot be obtained, The upper limit of the content, which is not particularly limited, is preferably 50% by mass or less.

### (B) Hydrophobically Modified Polyurethane

The hydrophobically modified polyurethane to be used in the skin cosmetic product of the present invention is preferably selected from associative thickening agents represented by the following general formula (I):

R₁-{(O-R₂)ₖ-OCONH-R₃[-NHCOO-(R₄-O)ₙ-R₅]ₕ}ₘ (I).

The associative thickening agent is a copolymer having a hydrophilic moiety as a skeleton and a hydrophobic moiety at the ends. It is considered that hydrophobic moieties of the copolymer are mutually associated in an aqueous medium and the hydrophilic moiety forms a shape like a loop or a bridge, thereby exerting a thickening effect.

In the above formula (1), R₁, R₃ and R₄ each independently represent a hydrocarbon group having 2 to 4 carbon atoms and preferably an alkyl group or alkylene group having 2 to 4 carbon atoms.

R₃ represents a hydrocarbon group, which may have a urethane bond, having 1 to 10 carbon atoms.

R₅ represents a hydrocarbon group having 8 to 36 and preferably 12 to 24 carbon atoms.

m is a number of 2 or more and preferably 2; h is a number of 1 or more and preferably 1; k is a number of 1 to 500 and preferably 100 to 300; and n is a number of 1 to 200 and preferably 10 to 100.

Examples of hydrophobically modified polyurethane particularly preferable in the present invention include a (PEG-240/Decyltetradeceth-20/HDI) copolymer (copolymer represented by the above formula (I), wherein R₁=an ethyl group, R₂ and R₄ each independently represent an ethylene group, R3=a hexamethylene group, R₅=a 2-dodecyldodecyl group, h=1, m=2, k=120, and n=20). The copolymer is commercially available from ADEKA CORP., under a trade name of “Adecanol GT700” or “Adecanol GT730”.

In the skin cosmetic product of the present invention, the content of a hydrophobically modified polyurethane relative to the total amount of cosmetic product is 0.5 to 3% by mass, 0.5 to 2.5% by mass, 0.5 to 2% by mass, or 0.5 to 1.5% by mass. If the content is less than 0.5% by mass, the (adipic acid/neopentyl glycol) crosspolymer powder cannot be stably blended in a water phase. In contrast, the content of more than 3% by mass is not preferable since the user sometimes feels sticky.

The skin cosmetic product of the present invention is preferably an aqueous cosmetic product increased in viscosity. The “aqueous skin cosmetic product” herein refers to a skin cosmetic product having an oil content of 20% by mass or less. If an oil is blended, the cosmetic is improved in compatibility with e.g., an oily cosmetic product to be applied thereon and the stability of a hydrophobic powder can be improved; however, the user sometimes feels heavy when the cosmetic product is applied because of the content of oil. The aqueous skin cosmetic product of the present invention, since it comprises a hydrophobically modified polyurethane (B), can stably blend an (adipic acid/neopentyl glycol) crosspolymer powder (A) in a large amount in a water phase, and thus, the content of oil can be reduced to 10% by mass or less and further 5% by mass or less. Because of this, the user can feel extremely light when the cosmetic product is applied. The content of oil can further be reduced to 3% by mass or less or a cosmetic product comprising no oil can be produced. Note that, when oil is blended, the lower limit of the oil content, which is not particularly limited, can be, for example, 0.1% by mass or more.

The oil component that can be blended in the skin cosmetic product of the present invention, which is an oily component usually blended in skin cosmetic products, may be one or two or more oily components selected from the group consisting of, for example, liquid and solid fats, waxes, hydrocarbons, higher fatty acids, higher alcohols, ester oils and fluorine-based oils, and is not particularly limited.

Specific examples thereof include liquid fats such as avocado oil, camellia oil, macadamia nut oil, mink oil, olive oil, castor oil, jojoba oil, triglycerin and glycerin trioctanoate; hydrocarbons such as liquid paraffin, squalene, paraffin, ceresin and squalene; higher fatty acids such as lauric acid, myristic acid, palmitic acid, stearic acid, behenic acid, oleic acid, 12-hydroxystearic acid, isostearic acid, linoleic acid and linolenic acid; higher alcohols such as lauryl alcohol, cetyl alcohol, stearyl alcohol, behenyl alcohol, oleyl alcohol, monostearyl glycerol ether, mono palmityl glycerol ether, cholesterol, phytosterol and isostearyl alcohol; ester oils such as coco-caprylate, isononyl isononanoate, isopropyl myristate, cetyl octanoate, octyldodecyl myristate, butyl stearate, decyl oleate, ethylene glycol dioctanoate, diisostearyl malate, trimethylolpropane trioctanoate, trimethylolpropane triisostearate, pentaerythritol tetraoctanoate, glyceryl trioctanoate, glyceryl triisostearate, ethyl acetate, butyl acetate and amyl acetate; straight-chain silicone Oils such as dimethyl polysiloxane, methyl phenyl polysiloxane, methyl hydrogen polysiloxane and diphenylsiloxy phenyl trimethicone; cyclic silicone oils such as decamethylcyclopentasiloxane, dodecamethylcyclohexasiloxane and octamethylcyclotetrasiloxane; and solid or semisolid fats at normal temperature such as yellow wax, candelilla wax, cotton wax, carnauba wax, bayberry wax, insect wax, whale wax, montan wax, rice bran wax, lanolin, kapok wax, Japan wax, lanolin acetate, liquid lanolin, sugarcane wax, isopropyl lanolate, hexyl laurate, reduced lanolin, jojoba wax, hard lanolin, shellac wax, bees wax, microcrystalline wax, paraffin wax, POE lanolin alcohol ether, POE lanolin alcohol acetate, POE cholesterol ether, polyethylene glycol lanolate, fatty acid glyceride, hydrogenated castor oil and vaseline and POE hydrogenated lanolin alcohol ether.

Note that the skin cosmetic product of the present invention is an aqueous cosmetic product having an oil content of at most 20% by mass or less, preferably 10% by mass or less and more preferably only 5% by mass or less, and therefore, heavy sense upon use due to oil components would not occur. Accordingly, unlike conventional oil-based irregularity correction cosmetic products, it is not necessary to blend e.g., a volatile silicone oil, for the purpose of mitigating the heavy sense of use. Thus, the skin cosmetic product of the present invention can be prepared as a cosmetic product comprising no silicone oil, i.e., so-called non-silicone (or silicone free) cosmetic product.

In the case of blending an oil, it is preferable to blend a water-soluble polymer having an emulsifying capacity together with the oil in order to further improve the stability of the cosmetic product. As the water-soluble polymer having an emulsifying capacity, an alkyl modified carboxyvinyl polymer can be preferably used. Specific examples thereof include an acrylic acid-methacrylic acid copolymer such as an (acrylates/C10-C30 alkyl acrylate) crosspolymer. As the water-soluble polymer, commercially available products such as Pemulen TR-1, Pemulen TR-2 (both are manufactured by BF Goodrich) may be used. The content of these water-soluble polymers preferably falls within the range of 0.01 to 0.5% by mass.

As long as the effect of the present invention is not damaged, components usually blended in cosmetic products and external preparations for skin, such as an ultraviolet absorber, a moisturizing agent, a fragrance, a surfactant, medicinal ingredients, a preservative, an antioxidant, a powder, a pigment, can be blended, if necessary.

The skin cosmetic product of the present invention can be produced in accordance with a method conventionally used in the art, for example, by separately mixing an aqueous component, a powder component, and an oily component and then adding the powder component and the oily component to the aqueous component, followed by stirring.

The form of the skin cosmetic product of the present invention may be a liquid, cream or gel, and preferably a gel having a viscosity of 15,000 mPa·s or more and 200,000 mPa·s or less.

The skin cosmetic product of the present invention can be provided particularly as an aqueous cosmetic product for correcting irregularities in a face or a body or preferably as e.g., a make-up foundation gel.

### Examples

Now, the present invention will be more specifically described by way of Examples; however, the present invention is not limited by these Examples. Contents are expressed by a unit of % by mass, unless otherwise specified.

Skin cosmetic products (samples) were prepared in accordance with the compositions shown in the following Tables 1 to 3. The samples of Examples were checked for the following items. The results are collectively shown in Tables 1 to 3.

### (1) Stable Blending of Powder

### “Stable Blending of Powder”

A: An (adipic acid/neopentyl glycol) crosspolymer powder was stably blended in a water phase during preparation.

C: An (adipic acid/neopentyl glycol) crosspolymer powder was not stably blended in a water phase (powder was isolated/precipitated).

### “Stability Over Time”

The samples containing an (adipic acid/neopentyl glycol) crosspolymer powder, which was successfully blended stably in a water phase during preparation, was stored at 50° C. for 2 weeks and then visually observed.

A: A sample was stable without powder isolation or water separation.

B: Powder isolation was not observed but water separated.

C: Powder was isolated/precipitated.

### (2) Irregularity Correction Effect

Experts (panelists) tested the samples by actually applying them on face and evaluated the samples in accordance with the following evaluation criteria.

A: Effect of correcting irregularities of skin such as pores is excellent.

B: Irregularities of skin such as pores are corrected (concealed) to some extent but not completely concealed.

C: No effect of correcting irregularities of skin is exerted.

### (3) Sebum Suppressing Effect

Three experts (panelists) tested the samples by actually applying them on face. Four hours later, the state of sebum was evaluated in accordance with the following evaluation criteria.

A: Shining due to sebum is not observed.

B: Shining due to sebum is low but observed.

C: Shining due to sebum is significantly observed.

TABLE 1

| | Compar- | Compar- | Compar- | Compar- | Compar- |
|---|---|---|---|---|---|
| | ative | ative | ative | ative | ative |
| | Exam- | Exam- | Exam- | Exam- | Exam- |
| | ple 1 | ple 2 | ple 3 | ple 4 | ple 5 |
| Deionized water | 58.4 | 56.2 | 56.75 | 46.75 | 56.6 |
| Propanediol | 8 | 8 | 8 | 8 | 8 |
| PEG-8 | 4 | 4 | 4 | 4 | 4 |
| Glycerin | 2 | 2 | 2 | 2 | 2 |
| Phenoxyethanol | 0.5 | 0.5 | 0.5 | 0.5 | 0.5 |
| Glycylglycine | 1.6 | 1.6 | 1.6 | 1.6 | 1.6 |
| Sodium acrylate | 0.5 | | — | | — |
| grafted starch | | | | | |
| Hydroxypropyl- | — | 0.5 | — | — | — |
| cellulose | | | | | |
| (Ammonium acryloyl- | — | 0.2 | — | — | — |
| dimethyltaurate/ | | | | | |
| Beheneth-25 meth- | | | | | |
| acrylate) crosspolymer | | | | | |
| (Acrylates/C10-30 | — | — | 0.1 | 0.1 | — |
| alkyl acrylate) cross- | | | | | |
| polymer (*1) | | | | | |
| (PEG-240/Decyltetra- | — | — | — | — | 0.3 |
| deceth-20/HDI) | | | | | |
| copolymers (*2) | | | | | |
| Polyvinyl acetate | — | 2 | 2 | 2 | 2 |
| emulsion (film- | | | | | |
| forming agent) | | | | | |
| (Adipic acid/neo- | 25 | 25 | 25 | 35 | 25 |
| pentyl glycol) cross- | | | | | |
| polymer powder (*3) | | | | | |
| Coco caprylate | — | — | — | — | — |
| Potassium hydroxide | — | — | 0.05 | 0.05 | — |
| Total | 100 | 100 | 100 | 100 | 100 |
| Stable blending of | C | C | A | A | C |
| powder | | | | | |
| Stability over time | — | — | B | B | — |
| Irregularity correction | — | — | A | A | — |
| effect | | | | | |
| Sebum suppressing | — | — | A | A | — |
| effect | | | | | |

(*1) PEMULEN TR-1 (manufactured by B F Goodrich)
(*2) ADEKANOL GT730 (manufactured by ADEKA CORP.)
(*3) PENSTIA POWDER (manufactured by CENTERCHEM Inc.)

TABLE 2

| | Exam- | Exam- | Exam- | Exam- |
|---|---|---|---|---|
| | ple 1 | ple 2 | ple 3 | ple 4 |
| Deionized water | 56.4 | 55.7 | 45.7 | 30.7 |
| Propanediol | 8 | 8 | 8 | 8 |
| PEG-8 | 4 | 4 | 4 | 4 |
| Glycerin | 2 | 2 | 2 | 2 |
| Phenoxyethanol | 0.5 | 0.5 | 0.5 | 0.5 |
| Glycylglycine | 1.6 | 1.6 | 1.6 | 1.6 |
| Sodium acrylate | — | — | — | — |
| grafted starch | | | | |
| Hydroxypropyl- | — | — | — | — |
| cellulose | | | | |
| (Ammonium acryloyl- | — | — | — | — |
| dimethyltaurate/ | | | | |
| Beheneth-25 meth- | | | | |
| acrylate) crosspolymer | | | | |
| (Acrylates/C10-30 | — | — | — | — |
| alkyl acrylate) cross- | | | | |
| polymer (*1) | | | | |
| (PEG-240/Decyltetra- | 0.5 | 1.2 | 1.2 | 1.2 |
| deceth-20/HDI) | | | | |
| copolymers (*2) | | | | |
| Polyvinyl acetate | 2 | 2 | 2 | 2 |
| emulsion (film- | | | | |
| forming agent) | | | | |
| (Adipic acid/neo- | 25 | 25 | 35 | 50 |
| pentyl glycol) cross- | | | | |
| polymer powder (*3) | | | | |
| Coco caprylate | — | — | — | — |
| Potassium hydroxide | — | — | — | — |
| Total | 100 | 100 | 100 | 100 |
| Stable blending of | A | A | A | A |
| powder | | | | |
| Stability over time | A | A | A | A |
| Irregularity correction | A | A | A | A |
| effect | | | | |
| Sebum suppressing | A | A | A | A |
| effect | | | | |

(*1) PEMULEN TR-1 (manufactured by B F Goodrich)
(*2) ADEKANOL GT730 (manufactured by ADEKA CORP.)
(*3) PENSTIA POWDER (manufactured by CENTERCHEM Inc.)

TABLE 3

| | | | | Compar- |
|---|---|---|---|---|
| | | | | ative |
| | Exam- | Exam- | Exam- | Exam- |
| | ple 5 | ple 6 | ple 7 | ple 6 |
| Deionized water | 45.55 | 51.15 | 51.15 | 78.4 |
| Propanediol | 8 | 8 | 8 | 8 |
| PEG-8 | 4 | 4 | 4 | 4 |
| Glycerin | 2 | 2 | 2 | 2 |
| Phenoxyethanol | 0.5 | 0.5 | 0.5 | 0.5 |
| Glycylglycine | 1.6 | 1.6 | 1.6 | 1.6 |
| Sodium acrylate | — | — | — | — |
| grafted starch | | | | |
| Hydroxypropyl- | — | — | — | — |
| cellulose | | | | |
| (Ammonium acryloyl- | — | — | — | — |
| dimethyltaurate/ | | | | |
| Beheneth-25 meth- | | | | |
| acrylate) crosspolymer | | | | |
| (Acrylates/C10-30 | 0.1 | 0.1 | 0.1 | — |
| alkyl acrylate) cross- | | | | |
| polymer (*1) | | | | |
| (PEG-240/Decyltetra- | 1.2 | 0.6 | 0.6 | 0.5 |
| deceth-20/HDI) | | | | |
| copolymers (*2) | | | | |
| Polyvinyl acetate | 2 | 2 | 2 | 2 |
| emulsion (film- | | | | |
| forming agent) | | | | |
| (Adipic acid/neo- | 35 | 30 | 25 | 3 |
| pentyl glycol) cross- | | | | |
| polymer powder (*3) | | | | |
| Coco caprylate | — | — | 5 | — |
| Potassium hydroxide | 0.05 | 0.05 | 0.05 | — |
| Total | 100 | 100 | 100 | 100 |
| Stable blending of | A | A | A | A |
| powder | | | | |
| Stability over time | A | A | A | A |
| Irregularity correction | A | A | A | B |
| effect | | | | |
| Sebum suppressing | A | A | A | B |
| effect | | | | |

(*1) PEMULEN TR-1 (manufactured by B F Goodrich)
(*2) ADEKANOL GT730 (manufactured by ADEKA CORP.)
(*3) PENSTIA POWDER (manufactured fay CENTERCHEM Inc.)

As is apparent from the results shown in Tables I to 3, a large amount (15% by mass) of (adipic acid/neopentyl glycol) crosspolymer powder was able to be stably blended by using 0.5% by mass or more of a hydrophobically modified polyurethane in combination. As a result, an extremely excellent irregularity correction effect and sebum suppressing effect were exerted (Examples 1 to 4). However, the powder was not stably blended if the content of the hydrophobically modified polyurethane was less than 0.5% by mass (Comparative Example 5).

Whereas, when a water-soluble thickening agent except the hydrophobically modified polyurethane was blended, the stability of the (adipic acid/neopentyl glycol) crosspolymer powder was inferior. For example, in the cases of using sodium acrylate grafted starch and hydroxypropylcellulose, the powder was not stably blended (Comparative Examples 1 and 2). In the case of acrylates/C10-30 alkyl acrylate crosspolymer, stable blending was successfully made during preparation; however, water was isolated after two weeks (Comparative Examples 3 and 4). However, in the case where a hydrophobically modified polyurethane was blended together, stability over time was drastically improved (Examples 5 and 6) and the stability did not decrease even if an oil was blended (Example 7).

Note that, when the (adipic acid/neopentyl glycol) crosspolymer powder was used in the content (3% by mass) recommended by the manufacturer, stable blending was successfully made; however, a sufficient irregularity correction effect and sebum suppressing effect were not obtained (Comparative Example 6).

What is claimed is:

1. A skin cosmetic product comprising:
(A) 15 to 50% by mass of a powder formed of an (adipic acid/neopentyl glycol) crosspolymer, and
(B) 0.5 to 3% by mass of a hydrophobically modified polyurethane.

2. The skin cosmetic product according to claim 1, wherein:
the hydrophobically modified polyurethane is (PEG-240/Decyltetradeceth-20/HDI) copolymer.

3. The skin cosmetic product according to claim 1, wherein:
the skin cosmetic product is a gel having a viscosity of 15,000 mPa·s or more and 200,000 mPa·s or less.

4. The skin cosmetic product according to claim 1, wherein:
the skin cosmetic is an aqueous cosmetic product having an oil content of at most 20% by mass or less.

5. The skin cosmetic product according to claim 3, wherein:
the skin cosmetic is an aqueous cosmetic product having an oil content of at most 20% by mass or less.
